(19) United States
(12) Patent Application Publication
(10) Pub. No.: US 2013/0289282 A1
(43) Pub. Date: Oct. 31, 2013

(54) THIN FILM TUBE REACTOR

(71) Applicant: Colin L. Raston, South Yunderup (AU)

(72) Inventor: Colin L. Raston, South Yunderup (AU)

(73) Assignee: THE UNIVERSITY OF WESTERN AUSTRALIA, Nedlands (AU)

(21) Appl. No.: 13/823,531

(22) PCT Filed: Sep. 12, 2011
(86) PCT No.: PCT/AU11/01168

§ 371 Date: Jul. 17, 2013

(30) Foreign Application Priority Data
Sep. 13, 2010 (AU) 2010904102

(51) Int. Cl. B01J 10/02; C07D 213/16

(52) U.S. Cl. CPC B01J 10/02; C07D 213/16
USPC 546/350; 422/202; 422/186.01; 422/186.29; 422/186.3; 422/127; 422/209; 422/186

(57) ABSTRACT

A thin film tube reactor comprising a tube having a longitudinal axis, an inner cylindrical surface, a closed end and an open end, wherein the tube is rotatable about the longitudinal axis and wherein the angle of the longitudinal axis relative to the horizontal is variable between about 0 degrees and about 90 degrees.

### FIELD OF THE INVENTION

The present invention relates to apparatus for mass transfer applications. More particularly, the present invention relates to thin film surface reactors.

### BACKGROUND ART

Mass transfer is one of the important unit operations in the field of process engineering and a variety of equipment has become available for all kinds of mass transfer applications. For example, packed bed, distillation columns and spargers are the conventional equipments for gas-liquid mass transfer.

Process intensification is a strategy employed to enhance transport properties by orders of magnitude by understanding the hydrodynamic behaviour of a system. The benefits include enhanced throughput, miniaturization of plant, higher quality of product and importantly, a reduction in the cost of production. However, there are only a few intensified modules that are available for mass transfer applications.

Examples of intensified modules include the spinning disc reactor (SDR) and the rotating tube reactor (RTR). As opposed to a batch reactor where a pool of reactants is mixed together all at once, these intensified modules create a thin film spread across a surface. The film thickness contributes to many influential chemical processing characteristics, one being a very high surface area to volume ratio. This high ratio makes for larger, more influential interactions between the film and its surroundings.

An SDR consists of a disc of diameter ranging from about 60 to about 500 mm and a surface that may be smooth, lined or meshed. The disc typically spins at around 1500 rpm or higher. Reactions performed on an SDR provide vigorous liquid film mixing with high transfer rates, extremely short reaction residence times enabling impulse heating and immediate subsequent cooling and plug flow identifying even mixing and transfer throughout the entire film.

An RTR is a hollow cylinder that rotates along its longitudinal axis. Liquid reactants enter one end of the rotating cylinder and the centrifugal forces form a homogeneous liquid film on the inner surface of the cylinder creating an annular flow. Products are collected at the opposing end. The thickness of the liquid film is a key parameter in determining the rate of mass transfer between the phases. Parameters which influence the rate of mass transfer include, without limitation: (1) liquid flow rate; (2) rotational speed of the cylinder; (3) gas flow rate; and (4) inner diameter of the cylinder.

It has recently been recognised that such intensified modules as the SDR and RTR may be employed for the manufacture of particles, such as nanoparticles, by way of precipitation from solution on the rotating surface. The use of a rotating surface allows precipitation of nanoparticles from viscous, supersaturated solutions through homogeneous nucleation aided by strong micromixing. Therefore, nanoparticles with a tight size distribution, uniform shape, specific phase, controlled agglomeration, and with and without defects may be manufactured in bulk, and without the problems of uncontrolled agglomeration that are associated with traditional stirred-tank reactors.

Precipitation from solution represents an inexpensive and simple method for producing nanoparticles. Thus, with the escalating use of nanoparticles and their commercial applications, there is an ever-increasing need for the development of improved intensified modules for the production of nanoparticles in addition to other mass transfer applications.

It has also been recognised that such intensified modules as the SDR and RTR may be employed for controlling organic reactions, in preparing compounds that are difficult or of low practical convenience using batch processing, and limit the amount of waste generated. There is an escalating need for the preparation of organic compounds, while minimising side reactions, generation of waste, minimising energy usage, and minimising negative impacts on the environment.

In addition, it has been recognised that such intensified modules as the SDR and RTR may be employed for probing the structure of matter, for example, but not limited to, the use of intense shearing to disassemble self organised systems in a controlled way, with implications in drug delivery applications and the structure of macromolecules and polymers in general.

The preceding discussion of the background art is intended to facilitate an understanding of the present invention only. The discussion is not an acknowledgement or admission that any of the material referred to is or was part of the common general knowledge as at the priority date of the application.

### DISCLOSURE OF THE INVENTION

The present invention provides a thin film tube reactor comprising a tube having a longitudinal axis, an inner cylindrical surface, a closed end and an open end, wherein the tube is rotatable about the longitudinal axis and wherein the angle of the longitudinal axis relative to the horizontal is variable between about 0 degrees and about 90 degrees.

Preferably, the angle of the longitudinal axis relative to the horizontal is variable between greater than 0 degrees and less than 90 degrees. The angle of the longitudinal axis relative to the horizontal may be varied during the course of a reaction.

The applicant has identified that the results and nanoparticle products recovered from SDR and RTR are not interchangeable due to differing sites and rates of nucleation and crystal growth. The applicant has made the important and surprising discovery that by creating a thin film tube reactor wherein the tube is at an angle of incline greater than 0 degrees from the horizontal, the specific centrifugal forces which create the thin film in the tube may be maintained, whilst shear force effects are introduced leading to superior mixing of fluid reactant and the production of nanoparticles with sites and rates of nucleation and crystal growth that can be different again from those produced in the SDR and RTR, and different effects on chemical reactions, and the structure of self organised systems, and macromolecules and polymers. Thus, for angles greater than 0 degrees, the inventors found there is instability in the fluid reactant in the rotating tube wherein these fluids can have laminar flow or turbulent flow, depending on at least the speed and angle of incline, and flow rate for continuous operations.

In addition, SDP and RTP operate strictly under continuous flow conditions requiring significant volumes of liquid to probe the effects of intense shearing within their dynamic thin films. This limitation can be overcome herein by operating at an angle relative to the horizontal using a tube closed at one end which can operate in batch mode for specific volumes while maintaining turbulent flow with associated shear forces or laminar flow. Advantageously, the present invention may be operated under either continuous flow or batch mode.

It will be appreciated that the specific angle of incline will be chosen in accordance with the particular process being carried out using the thin film tube reactor of the present invention. In this respect, the greater the angle of incline of the longitudinal axis of the tube reactor relative to the horizontal, the greater the shear forces that will be created for the mixing of reactants in the thin film of the reactor. Thus, it will be appreciated that processes using the thin film tube reactor that benefit from, or require greater shearing forces to provide additional mixing or faster mixing, can employ a greater angle of incline of the longitudinal axis relative to the horizontal.

For the purposes of the present invention, it is preferable that the angle of incline relative to the longitudinal axis is between about 0 and about 60 degrees from horizontal in order to utilise the shearing forces in combination with the centrifugal forces to mix reactants within the thin film formed when using the tube reactor. More preferably, the angle of incline relative to the longitudinal axis of the tube reactor is between about 40 and about 50 degrees from horizontal, and even more preferably, the angle of incline relative of the longitudinal axis for the tube reactor is about 45 degrees from horizontal, including for, but not limited to, operating the invention under batch mode. The angle of incline for continuous flow processing can be less than for batch operation, typically, but not limited to, 5 degrees.

The tube of the thin film tube reactor may be provided in any cylindrical form. For example, the tube may be an elongate tube, such as a nuclear magnetic resonance (NMR) tube. Alternatively, the tube may have a radius that is greater than its length.

The tube of the thin film tube reactor may be substantially cylindrical or may comprise at least a portion that is tapered. In one form of the invention, the whole tube is tapered.

Further, the tube of the reactor may comprise a lip adjacent to the open end. An advantage of the lip includes that fluid reactant can be inhibited from exiting the open end of the tube when this is not desired.

The rotation of the tube about its longitudinal axis is required to generate sufficient centrifugal forces to create the thin film of reactant in the tube during a process using the present invention. Thus, in a preferred aspect, the present invention comprises a means for rotating said tube around its longitudinal axis. Preferably, the speed of rotation of said tube around the longitudinal axis is variable. Thus, a speed of rotation will be chosen as is preferential for a process using the present invention. A motor may be used for providing said rotation of the tube, more preferably, a variable speed motor. Said motor may comprise a drive shaft attached to the tube reactor to cause the tube to rotate. Rotational speeds of 15000 rpm or higher for the tube are possible with the present invention. The rotational speed can be varied and adjusted, and the frequency of this change in speed can be adjusted, from a fraction of a second or higher.

The speed of rotation may be varied during processing, and varied periodically at a set frequency and amplitude of change in speed.

The rotation of said tube causes reactant introduced into the tube to form a thin film on the inner cylindrical surface of the tube. Thus, the thin film tube reactor comprises means for supplying at least one fluid reactant to the tube. The means may comprise one or more feed tubes. Where the thin film tube reactor comprises more than one feed tube, said feed tubes may comprise feed tubes of variable lengths. In this respect, fluid reactant may be introduced into the tube reactor at different locations. As a result, several reactions may be telescoped within a single tube and tube reactor of the invention.

The open end of the tube comprises the end of the tube through which reactant may be introduced and/or removed. In this regard, a portion of the open end of the tube may be covered provided that there is a means for introducing and/or removing reactant from the tube reactor.

The tube of the thin film tube reactor may comprise various surface structures or aberrations on the inner surface for breaking down the boundary layer of at least one reactant. Such structures may comprise, in some non-limiting examples, channels, a structured mesh surface or pits or may be in the form of depressions on the inner surface or extensions from the inner surface. Here, such structures or aberrations may be used, for example, for immobilising a reaction catalyst on the inner surface.

The thin film tube reactor may also comprise means for introducing a gas to the tube. Moreover, a means for removing an unwanted gas from the tube may also be provided. The means for introducing or removing a gas may comprise one or more gas feed tubes. Said gas feed tubes may or may not rotate. Where the gas feed tubes rotate, they may rotate at the same rate as the tube or at a different rate. Thus, the present invention provides that reactions may be carried out inside the tube in an inert atmosphere of, for example, nitrogen or argon, or reactive gases, for example, hydrogen, carbon dioxide, carbon monoxide and anhydrous ammonia, either through gas feed or by placing the tube reactor in a controlled atmosphere environment.

The tube of the reactor may comprise a jacket surrounding at least a portion of the tube. Said jacket may be used to provide heating and/or cooling for the tube. For example, amongst others, the jacket may be a heat transfer jacket of the type comprising inductive, resistive, conductive and heat transfer fluid. The jacket may provide insulation for the tube against the external environment of the thin film tube reactor. Moreover, a plurality of jackets may be employed for the tube reactor, each jacket configured to increase, decrease or insulate the process temperature along the length of the tube.

The thin film tube reactor may further comprise a cooled gas from a liquid nitrogen source directed at a portion of the tube. The thin film tube reactor of the present invention may further comprise an electromagnetic source directed at a portion of the tube. The thin film tube reactor of the invention may comprise other field effects, for example, UV, different wavelength laser radiation, magnetic, microwave, acoustic and sonic energy. Some of these field effects may be provided through the drive shaft.

The thin film tube reactor of the present invention may comprise means for removing a product from the tube. Preferably, said means comprises a collector positioned substantially adjacent to the open end of the tube for collecting product from the tube. Said collector may comprise a product inlet for collecting product from the tube. The collector may comprise a reservoir component. The product may enter the collector under centrifugal force. The collector itself may rotate at the same speed or at a different speed to the speed of rotation of the tube around the longitudinal axis. The reservoir component may comprise an end plate with a plurality of radially spaced product outlets that have a radial position such that a known product may exit the reservoir component through each of the outlets under centrifugal force. Alternatively, the collector may comprise a single product outlet for removing product from the collector and therefore the tube reactor. Product may exit the single product outlet under centrifugal force.

The collector may comprise a filtration membrane. Such filtration may be used to separate product from solution. Some non-limiting examples of such filtration membranes comprise a crossflow filtration membrane, a dead-end filtration membrane, an ultrafiltration membrane, a reverse osmosis membrane and a nanofiltration membrane.

In certain circumstances reaction product will need to be maintained at a low or even a high temperature compared to the temperature of the environment surrounding the thin film tube reactor. The collector may comprise a cooling and/or heating and/or insulation system for maintaining an appropriate temperature of the reaction product as is required. This system may be in the form of a passage in the wall of the collector which reaction product under centrifugal force is forced against. In one non-limiting example, coolant may be introduced into the passage through an inlet into the wall of the collector, where it can absorb heat from the reaction product on the other side of the wall of the passage and then exit the passage through an outlet from the collector. Such control of parameters such as temperature enables high reproducibility of results with the present invention through more efficient heat dissipation. In addition, rapid heating on the tube and cooling during exit of the fluid reactant is possible, unlike traditional batch reactions, thereby minimising the potential for side reactions and providing precise control of reaction parameters.

In another aspect of the thin film tube reactor of the present invention, the closed end of the tube comprises a mixing plate rotatable around the longitudinal axis. The mixing plate may comprise a mixing plate of a spinning disc reactor. Thus, the tube may comprise a cylindrical tube with a mixing plate that is substantially circular and coaxial with said tube. Alignment or connection between the mixing plate and tube where an outer edge of said mixing plate contacts the inner surface of the tube would result in a closed end of the tube, such that reactant cannot exit the tube between outer edge of mixing plate and inner edge of tube.

In another aspect of the invention, the location of the mixing plate in the tube is variable along the longitudinal axis of the tube. This variable position may include movement of the mixing plate through the tube along the longitudinal axis of the tube during a process using the thin film tube reactor of the invention.

The mixing plate may comprise surface structures to affect the hydrodynamics of the at least one reactant. Such surface structures may comprise, for example, corrugations. In this respect, such corrugations may also be present on the inner surface of the tube.

The thin film tube reactor of the present invention may be configured such that it can process at least one reactant in a continuous flow operation. Alternatively, the reactor may be configured for re-circulating at least one reactant or configured for operating substantially in a batch mode. Importantly, the angle of incline of the tube relative to horizontal enables batch processing to be possible where a steady state of reactant may be maintained in the tube. This may be used in studying the shearing of self-organised systems and applications thereof. Such a batch system may be robotically controlled to produce a sequential batch system, useful in large scale production. Thus, the reactor provides for scaling up and scaling down of such processes. The presence of a lip adjacent to the open end of the tube assists in maintaining reactant in the tube where the angle of inclination of the tube from the horizontal is small, particularly during batch processing.

Rotation of the tube can create a vortex at or near the closed end of the tube. It is possible to control the location of the vortex relative to the closed end by varying the speed of rotation of the tube, the volume of reactants and the angle of inclination of the tube. Where the vortex is not fully developed to the closed end of the tube, different mixing zones can result and these can be used to fabricate materials containing different properties, depending of the mixing zone where the materials formed. For example, bimodal polymers may prepared. Where the vortex is fully developed to the closed end, the non-partition thin films can be used to prepared materials based on the predominance of product with uniform properties.

The tube may be removable from the thin film tube reactor of the present invention. This is a further advantage over traditional horizontal continuous flow tube reactors, enabling the removal of the tube containing fluid reactant for analysis. Such analysis including, for example, nuclear magnetic resonance (NMR) can allow the study of reactions, particularly in the batch mode. The disassembly of molecular capsules within the thin films, for encapsulation of molecules, for example, can be monitored by NMR. Significantly, the tube of the reactor being removable provides for replacement with alternative tubes of any material. In some non-limiting examples, the tube may comprise: quartz for UV studies which can be used for in situ online real time analysis of the reactions and processing in the tube; Teflon; stainless steel; tubes with the inner surface coated, for example with catalysts; and inner surfaces comprising structural features for enhanced mixing and shearing rates.

Another advantage of being able to remove the tube from the reactor is for ease of determining the average film thickness of a fluid in the tube for a specific angle of incline from the horizontal and speed, especially when the vortex is fully developed to the closed end of the tube. This is determined by simply weighing the tube after steady state, when no more fluid exits the tube, for a tube of known diameter, and thus known internal surface area, with the weight of fluid translating to a volume for a known density. It will be appreciated that the film thickness will be influenced by the location of the vortex. This is not possible for fixed horizontal continuous flow tube reactors.

In addition, being able to remove the tube from the reactor allows for ease of cleaning the tube, and carry out reactions in other tubes, for high through-put optimisation of reaction conditions. It will be appreciated that this can be extended to the use of robotic systems for automation of reactions for a large number of tubes.

A further advantage of the tube reactor of the present invention over traditional horizontal continuous flow tube reactors is that mixing rates of fluid reactant are usually faster. The increase in pressure within the thin films under continuous flow, batch or batch recycling, may enhance chemical reactions, for example Diels Alder reactions, which have a negative volume of activation. In addition, the intensity of shearing resulting in higher collision frequency and energy transfer may enhance chemical reactions, for example the doubling of the rate of dimerisation of cyclopentadiene for a tube inclined at 45 degrees for 1 mL of material, in a 10 mm OD tube rotated at 7000 rpm. Further, the present invention provides a high mass transfer of gases into the fluid reactant due to the breakdown in surface tension. It also provides a safe way of handling hazardous materials in that minimum quantities are required and the short residence time provides that fluids above their boiling points may be handled by the present invention.

The thin film tube reactor of the present invention may be configured for a mass transfer process selected from the group comprising, but not limited to, a heat treatment process, an emulsion-forming process, a suspension-forming process, and a chemical reacting process.

In a preferred aspect of the invention, the tube reactor is configured for a process for the fabrication of nanomaterials including nanoparticles. Thus, the thin film tube reactor of the present invention as described herein may be used to fabricate nanoparticles. High throughput in developing a library of nanomaterials and organic compounds is therefore possible using the thin film tube reactor of the present invention. This is possible through intense mixing associated with rotational speeds of 15000 rpm or more.

With respect to the fabrication of nanoparticles, in an embodiment of the present invention additional vibrational energy may be applied to the thin film on the inner surface of the rotating tube. Vibrational energy may be provided, for example, amongst others, by ultrasonic means, which may also be used to enhance organic and inorganic reactions and be used to disassemble material. Moreover, vibrational energy may be applied to product that has exited the rotating tube so as to reduce agglomeration of precipitated nanoparticles and for control chemical processes.

Electromagnetic radiation comprising, for example, amongst others, ultraviolet, infrared, X-ray, gamma ray, and light, and magnetic, and electric fields, may also be applied to the thin film on the inner surface of the rotating tube or to product that has exited the rotating tube so as to reduce agglomeration of precipitated nanoparticles and for controlled chemical reactions and assemble-disassembly processes.

Thus, in accordance with embodiments of the present invention described herein, there is provided the means for large-scale production of nanoparticles under continuous flow, or small scale for batch mode, as a ‘bottom up’ fabrication where nucleation and growth of particles occurs in a controlled way. The shape, form, size and size distribution of nanoparticles precipitated from the present invention can be controlled through adjustment of reaction conditions comprising, for example, amongst others: angle of incline relative to horizontal; control of the speed of rotation; the shape, configuration and material of the tube of the device; temperature; pressure; vibrational and/or electromagnetic energy, and magnetic and electric fields.

Even though the present invention has been described as having use in producing nanoparticles, it is not limited thereto. In this regard, large particles may also be produced, and the invention can be used also for, but not limited to, controlling chemical reactions in making molecules, and inorganic complexes, and controlling the organisation of matter involving shear forces in the thin films. This also includes the ‘top down’ fabrication of nano-materials.

The preferred angle of inclination will be determined, at least in part, by the requirement of a vortex being developed all the way to the closed end or partly developed, and whether the reactor is to be operated in a continuous flow or in batch mode. Modes of operation depending on the variable angle, θ, include
1. Continuous flow mode—vortex not developed to the closed end of the tube.
2. Continuous flow mode—vortex developed to the closed end of the tube.
3. Batch mode—vortex not developed to the closed end of the tube.
4. Batch mode—vortex developed to the closed end of the tube.
5. Sequential use of two reactors, operating under the two continuous flow mores (1. and 2.) and variances thereof.

### BRIEF DESCRIPTION OF THE DRAWINGS

FIG. 1 is a longitudinal cross-sectional view of a thin film tube reactor according to a first embodiment of the invention;

FIG. 2 is a cross-sectional view of the fluid collector of the thin film tube reactor of FIG. 1;

FIG. 3 is a cross-sectional view of a thin film tube reactor according to a second embodiment of the invention;

FIG. 4 is a cross-sectional view of a thin film tube reactor according to a third embodiment of the invention;

FIG. 5 is a plot of average film thickness versus rotation speed;

FIG. 6 is a graph representing the results of variable speed studies using the thin film tube reactor according to a first embodiment of the invention;

FIG. 7 is a graph representing the results of variable pass studies using the thin film tube reactor according to a first embodiment of the invention;

FIG. 8 is a graph representing the results of variable temperature studies using the thin film tube reactor according to a first embodiment of the invention;

FIG. 9 is a plot of room temperature dimerisation of cyclopentadiene; and

FIG. 10 is SEM images of salbutamol sulfate nanoparticles.

### DETAILED DESCRIPTION OF PREFERRED EMBODIMENTS OF THE INVENTION

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Reference to information contained in the text should not be understood as a concession that the material or information was part of the common general knowledge or was known in Australia or any other country.

Features of the invention will now be discussed with reference to the following non-limiting description and examples.

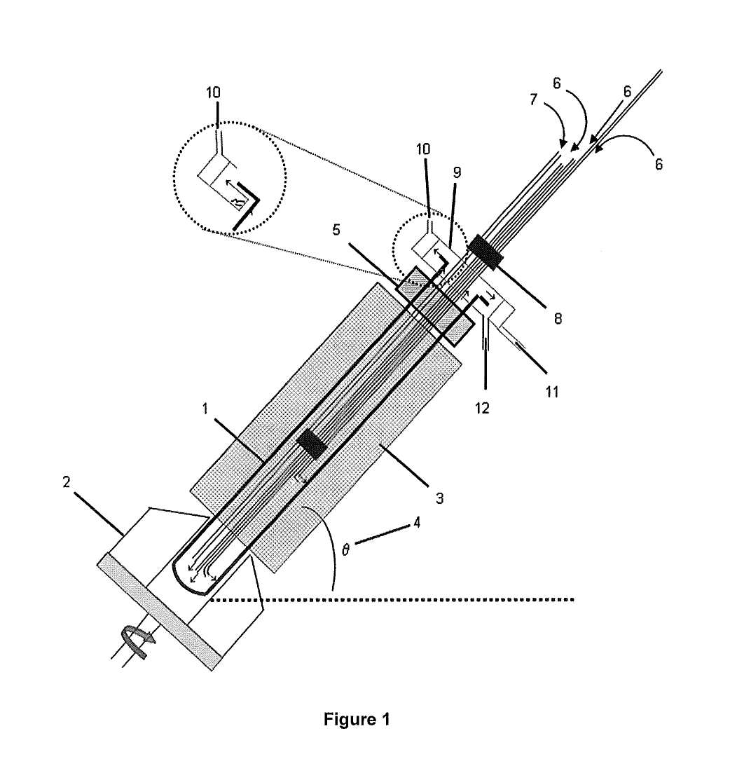

In a first non-limiting embodiment, a thin film tube reactor of the invention is illustrated in FIG. 1 and comprises a tube 1 rotatable about its longitudinal axis by a motor 2. The motor 2 can be a variable speed motor for varying the rotational speed of the tube 1. A jacket 3 can be used to partially or wholly surround the circumference of the tube 1 for heating and/or cooling and/or insulating the tube 1. The jacket 3 may also insulate the tube 1 from the external environment. The tube 1 is situated on an angle of incline 4 relative to the horizontal of between greater than 0 degrees and less than 90 degrees. The angle of incline 4 can be varied, and the speed of the motor can be varied and operated in controlled set frequency and set change in speed.

A spinning guide 5 assists in maintaining the angle of incline 4 and a substantially consistent rotation around the longitudinal axis of the tube 1.

Means for supplying at least one fluid reactant to the inner surface of the tube 1 can include feed tubes 6. These feed tubes 6 may be of varying lengths to supply fluids to variable locations on the inner surface of the tube 1. A gas feed tube 7 can supply gas to the tube 1 as is required for processes using the thin film tube reactor of the invention. One or more clamps 8 may be employed to hold feed tubes 6 and gas feed tubes 7 in position within the tube 1.

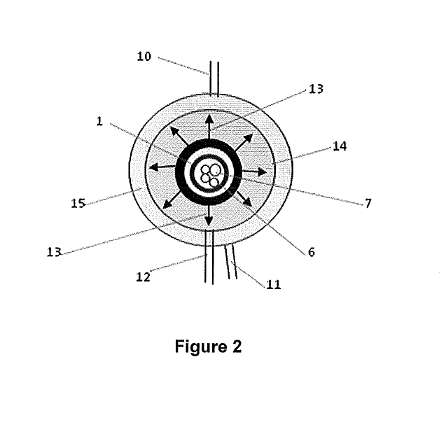

A collector 9 positioned substantially adjacent to the opening of the tube 1 can be used to collect fluid from the tube 1. An inlet 10 enables coolant to be introduced into the collector 9 as is required for processes using the device of the invention. Further, an outlet 11 enables coolant to be removed from the collector 9. The collector 9 may also comprise a product outlet 12 for collecting fluid product exiting the tube. In this respect, FIG. 2 illustrates that fluid product exiting the tube 1 may migrate (see arrows 13) under centrifugal force to the wall 14 of the collector 9, where it can exit through the product outlet 12. Coolant may enter the inlet 10 into a passage 15 within the walls of the collector 9. Coolant can exit the passage 15 through an outlet 11.

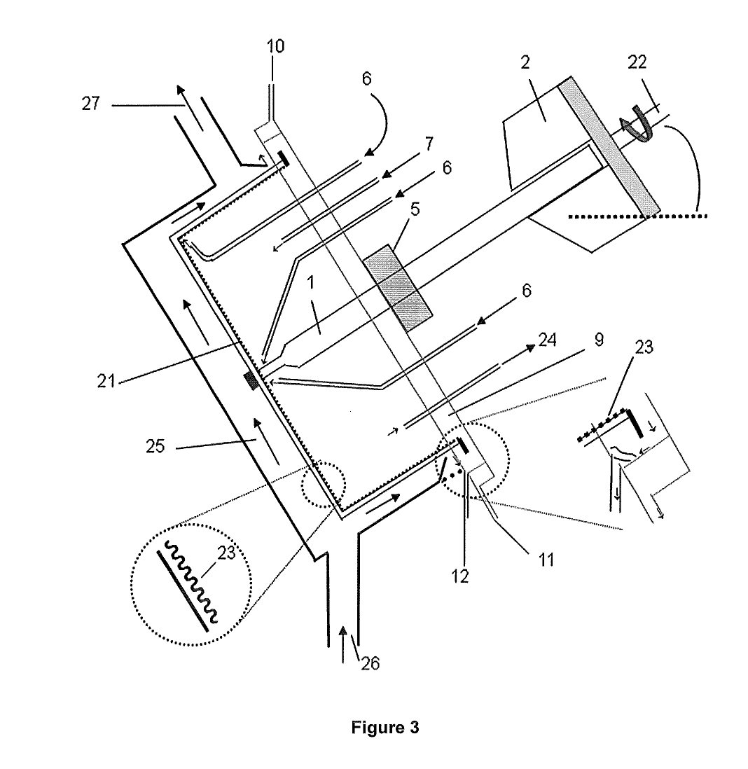

In a second embodiment of the invention illustrated in FIG. 3, the closed end of the tube of the thin film tube reactor comprises a mixing plate 21 of a spinning disc reactor. In this embodiment the mixing plate 21 forms the closed end of the tube 1. A motor 2 connected to a drive shaft 22 rotates the tube 1 about its longitudinal axis. The fluid feeds 6 may direct fluid reactant onto either or both the inner surface of the tube 1 or directly onto the mixing plate 21. The mixing plate 21 and the inner surface of the tube 1 may comprise corrugations 23 or may be substantially smooth. A gas outlet 24 may provide means for removal of gases. The walls of the tube and mixing plate of this embodiment may also comprise a passage 25 wherein heating or cooling air may be introduced in an inlet 26 and removed from the passage 25 via an outlet 27. The collector 9 may be configured with the tube 1 such that it substantially covers the open end providing a closed system.

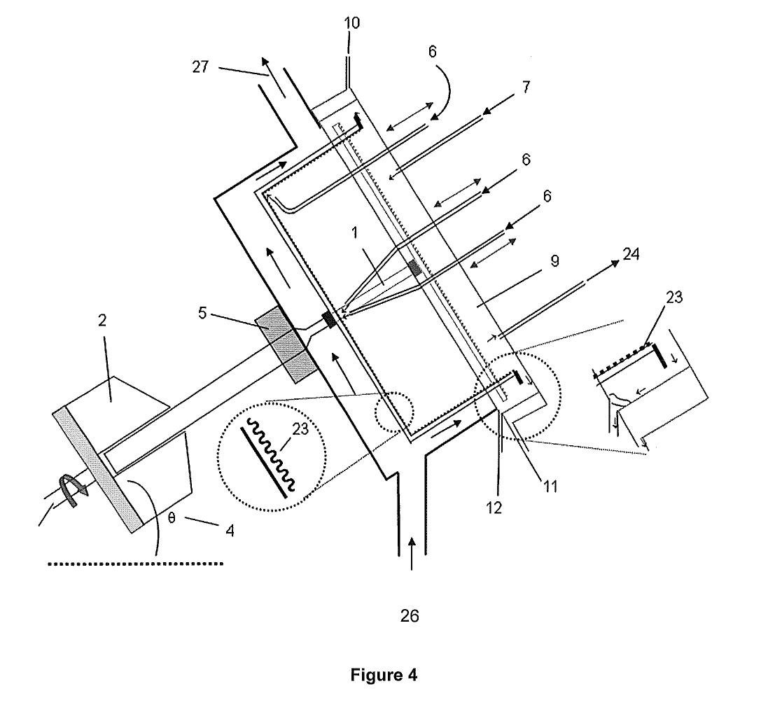

In a third embodiment of the invention illustrated in FIG. 4, a mixing plate 21 forms the closed end of the tube 1 similarly to the second embodiment. However, this embodiment provides for a variable position of the mixing plate 21 along the longitudinal axis of the tube 1. Thus, the motor 2 is placed behind the mixing plate 21 and the mixing plate can move, during use if required, along the drive shaft 22.

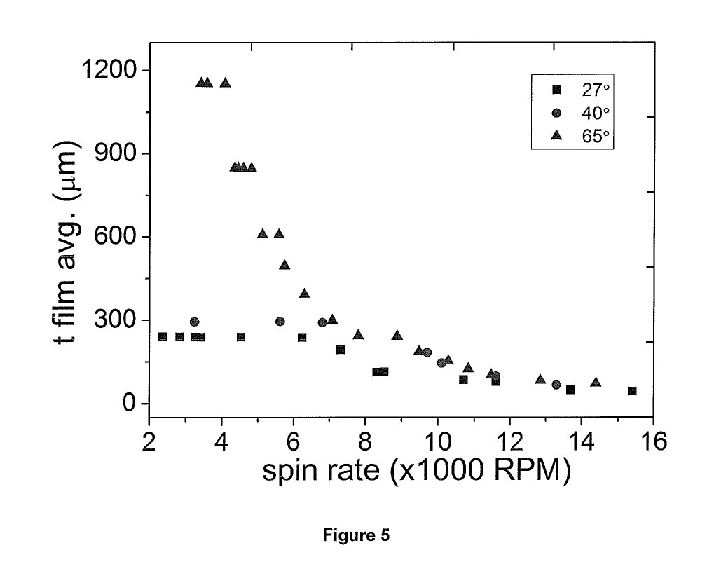

By changing the angles of inclination and the speed of rotation, it is possible to control the average film thickness of a fluid in the tube. FIG. 5 is a plot of average film thickness versus rotation speed for different angles of inclination for a vortex fully developed to the closed end and where the liquid film is fully extended to the open end.

### Disassembly and Organisation of Molecular Capsules

The thin film tube reactor operating at an angle of 45° relative to the horizontal axis, using a 10 mm NMR tube is effective in disassembling molecular capsules based on two p-phosphonated calix[5]arenes, as shown in Scheme 1. The average film thickness at 1500 rpm was 300 μm and over a five minute period aqueous solutions of carboplatin and the calixarene (1:2 molar ratio) resulted in dramatic changes in the ¹H chemical shifts for the methylene protons of the cyclobutane ring in carboplatin. This is consistent with the expected deshielding on encapsulation of a single drug molecule in a molecular capsule based on two calixarene molecules. For speeds less that 1500 rpm there was no evidence for encapsulation of carboplatin over the same 5 minute period and speeds higher than 1500 rpm were effective in encapsulating the carboplatin. ¹⁹⁵Pt NMR spectra show 85% encapsulation of the drug molecule, whereas traditional batch stirring of the same solution for five hours resulted in only 15% encapsulation, and sonication for at least five minutes resulted in 30% encapsulation. Concentration of solution results of the capsule encapsulating the drug results in its spontaneous release and the formation of amorphous material. Therefore, 1500 rpm for a 10 mm tube inclined at 45° corresponds close to the transition from laminar flow to a turbulent flow for the film, which is then responsible for the shear forces in disassembling the molecular capsules.

It has been established that a water soluble cavitand has unique self assembly properties in forming molecular capsules displaying fivefold symmetry, which can confine the anti-cancer drug carboplatin. The integrity of the capsules is altered through external forces in dynamic thin films in the invention.

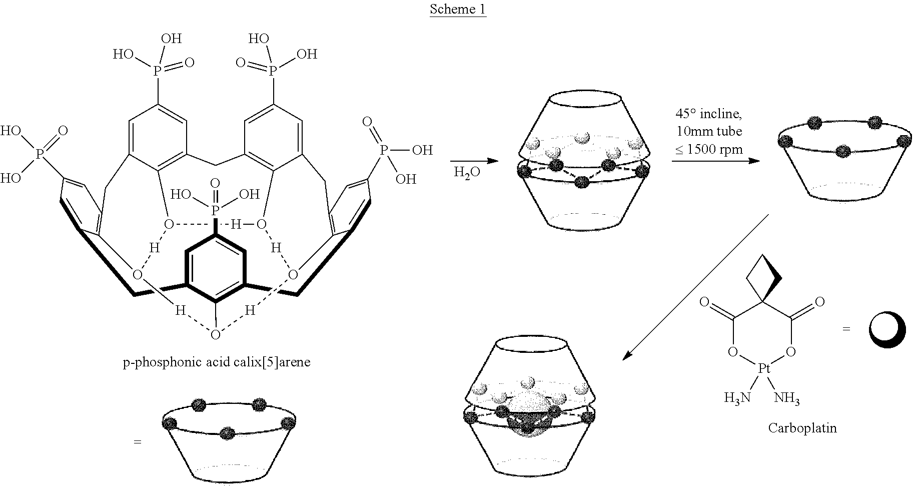

¹H, ¹⁹⁵Pt and ROESY experiments were performed on a Bruker 600 MHz NMR spectrometer. ¹⁹⁵Pt and ROESY experiments were performed without spinning, and ¹⁹⁵Pt experiments were performed over the course of 60 hours. DOSY experiments were carried out on a Bruker 500 MHz spectrometer without spinning.

### Preparation of Small Molecules

It is known to prepare triarylpyridines via Claisen-Schmidt condensation of a ketone and an aldehyde followed by Michael addition of the 1,5-dione as shown below in Scheme 2.

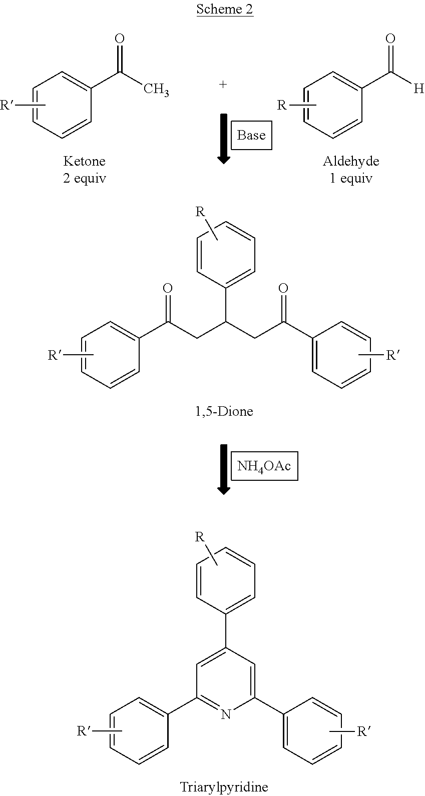

However, the condensation reaction is problematic and results in the formation of significant amounts of chalcone as shown below in Scheme 3.

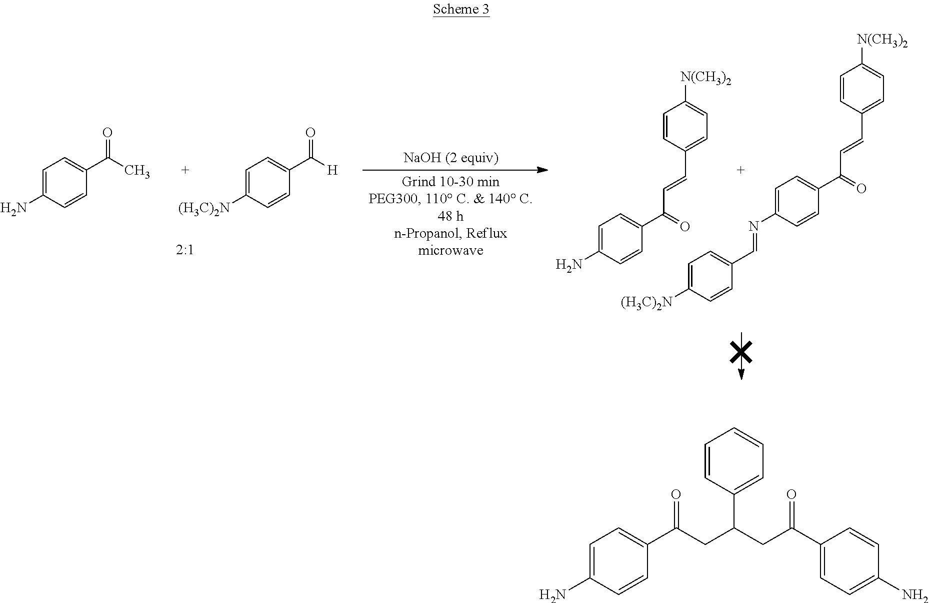

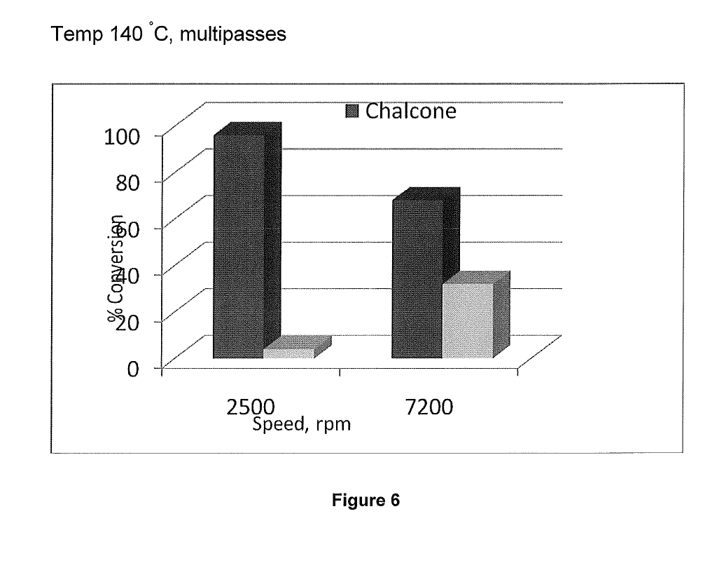

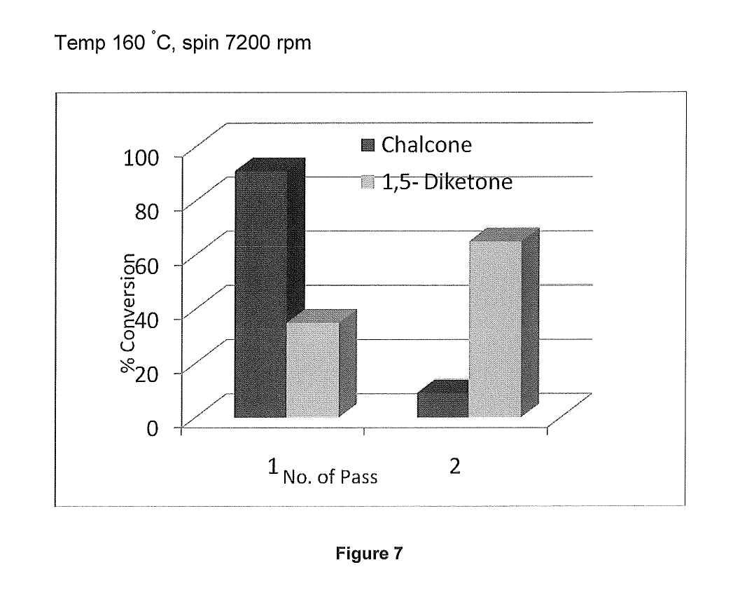

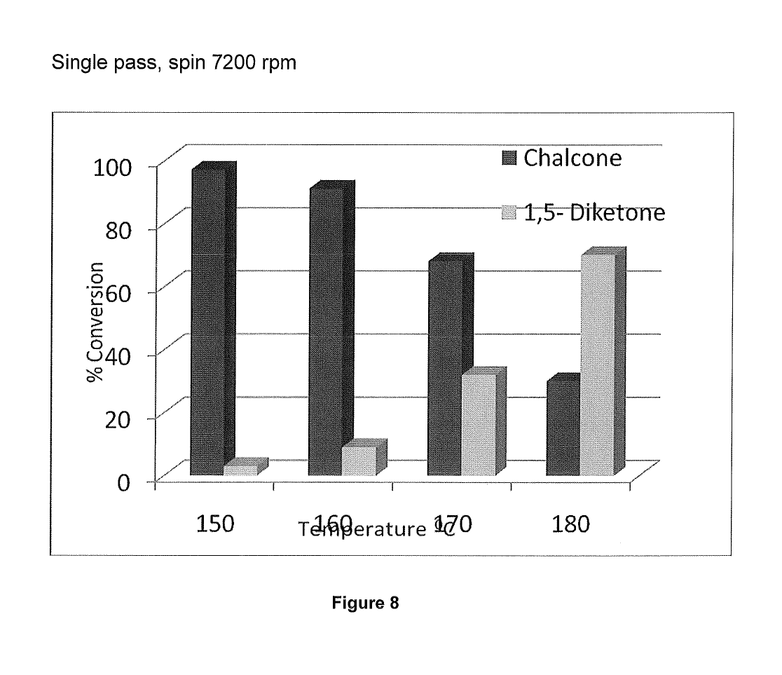

The applicant has discovered that the condensation reaction can proceed efficiently in the thin film tube reactor of the present invention as shown below in Scheme 4. FIGS. 6 to 8 present the results of reactions under various conditions showing that it is possible to control the 1,5-diketone/chalcone ratio using the thin film tube reactor of the present invention.

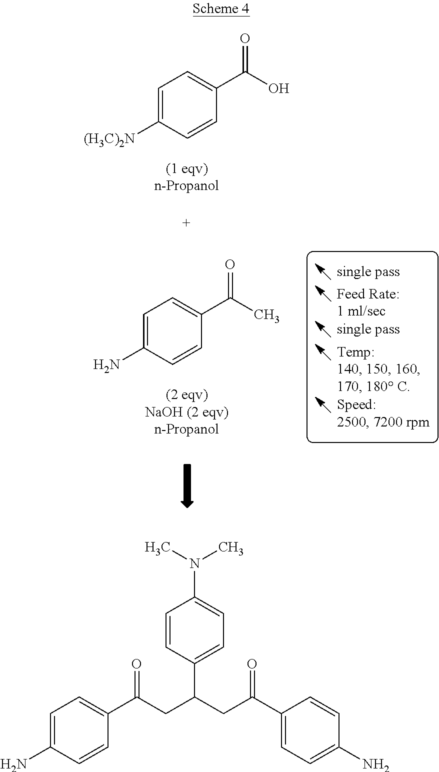

The condensation of resorcinol and pyrogallol with aromatic aldehydes under turbulent and continuous flow conditions in rotating tube reactor results in selective and direct formation of the resorcin[4]arene, R=H, and pyrogallol[4] arenes, R═OH, as the bowl shaped C₄ᵥ isomer rather than the C₂ₕ isomer as the first formed product using traditional batch processing (Scheme 5).

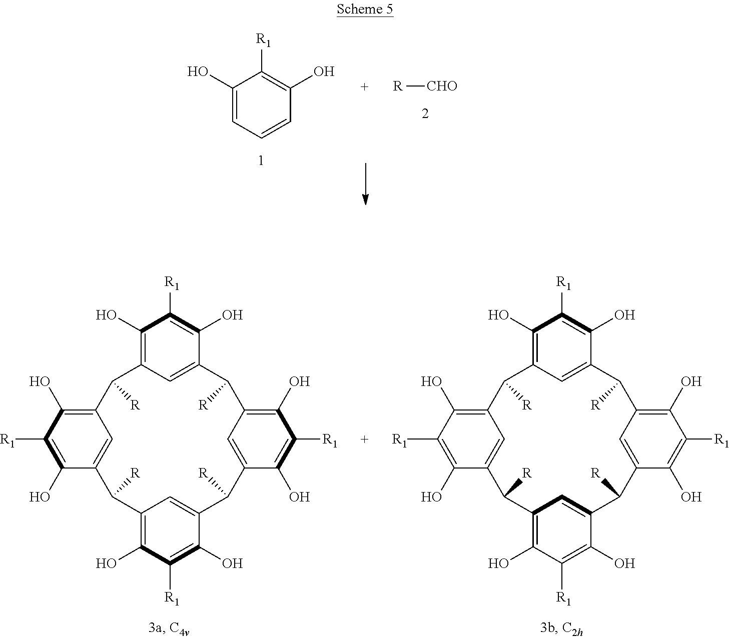

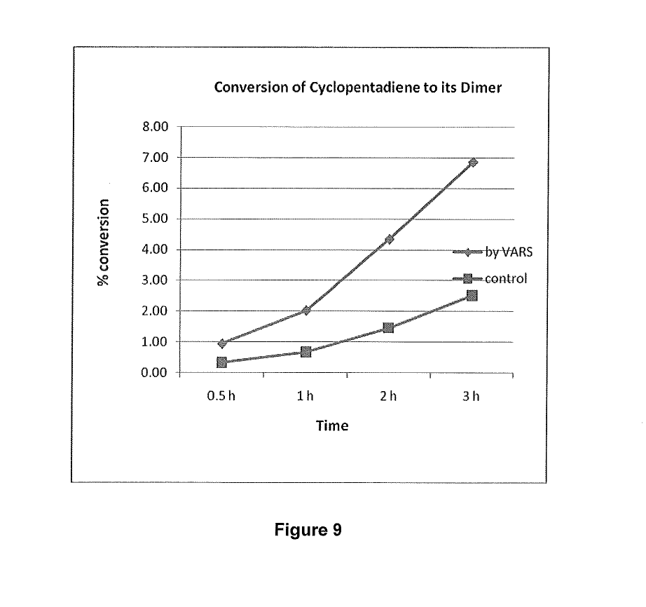

Cyclopentadiene is known to dimerise via a reversible Diels-Alder reaction. At room temperature, the conversion takes hours. FIG. 9 shows that the room temperature dimerisation of cyclopentadiene can be significantly accelerated using the thin film reactor operating at 45° and 7000 rpm

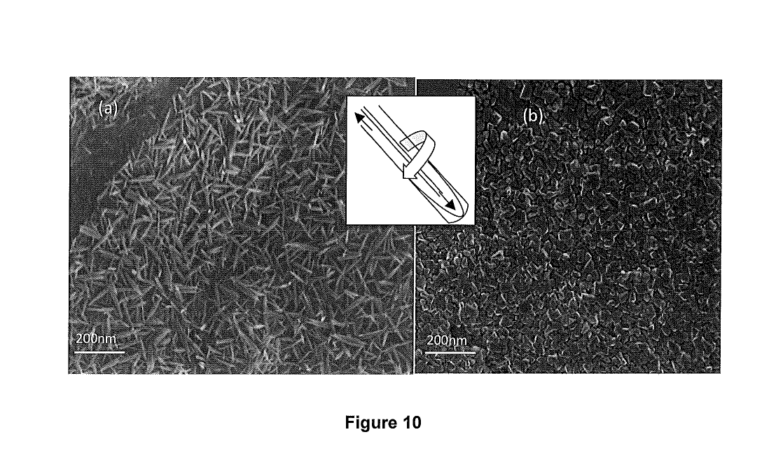

It is possible to control the size and shape of nanoparticles utilizing the apparatus of the present invention. FIG. 10 presents SEM images of different size and shape nano-particles of salbutamol sulfate, prepared under continuous flow conditions, at an inclination of 45°, where the vortex is fully developed to the closed end of the tube in the said device. Conditions: (a) 0.5 M sulfuric acid 10 mg/ml salbutamol base IPA 2000 rpm, flow rate 4 ml/min, and (b) 2 M sulfuric acid 20 mg/ml salbutamol base IPA, 2000 rpm, flow rate 4 ml/min.

1. A thin film tube reactor comprising a tube having a longitudinal axis, an inner cylindrical surface, a closed end and an open end, wherein the tube is rotatable about the longitudinal axis and wherein the angle of the longitudinal axis relative to the horizontal is variable between about 0 degrees and about 90 degrees.

2. A thin film tube reactor according to claim 1, wherein the angle of the longitudinal axis relative to the horizontal is variable between greater than 0 degrees and less than 90 degrees.

3. A thin film tube reactor according to claim 1, wherein the tube is substantially cylindrical or comprises at least a portion that is tapered.

4. A thin film tube reactor according to claim 1, wherein the tube comprises a lip adjacent to the open end.

5. A thin film tube reactor according to claim 1, wherein the speed of rotation of the tube about the longitudinal axis is variable.

6. A thin film tube reactor according to claim 1, wherein the thin film tube reactor comprises means for supplying at least one reactant to the tube.

7. A thin film tube reactor according to claim 1, wherein the inner surface of the tube comprises surface structures or aberrations.

8. A thin film tube reactor according to claim 1, wherein the thin film tube reactor comprises means for introducing a gas to the tube.

9. A thin film tube reactor according to claim 1, wherein the thin film tube reactor comprises means for removing a gas from the tube.

10. A thin film tube reactor according to claim 1, wherein the reactor comprises at least one jacket surrounding at least a portion of the tube, adapted to provide heating and/or cooling to the tube.

11. A thin film tube reactor according to claim 1, wherein the reactor comprises means to provide field effect such as UV, different wavelength laser radiation, magnetic, microwave, acoustic and sonic energy.

12. A thin film tube reactor according to claim 1, wherein the reactor comprises means for removing product from the tube.

13. A thin film tube reactor according to claim 12, wherein the means for removing product comprises a cooling and/or heating system.

14. A thin film tube reactor according to claim 1, wherein the closed end of the tube comprises a mixing plate rotatable around the longitudinal axis.

15. A thin film tube reactor according to claim 14, wherein the mixing plate is a mixing plate of a spinning disc reactor.

16. A thin film tube reactor according to claim 14, wherein the location of the mixing plate in the tube is variable along the longitudinal axis of the tube.

17. A mass transfer process using the thin film tube reactor according to claim 1.

18. A process for fabricating nanomaterials and micromaterials using the thin film tube reactor according to claim 1.

19. A process for controlling chemical reactions, including but not limited to preparing small molecules and polymers, using the thin film tube reactor according to claim 1.

20. A process for probing and controlling the structure of matter, including laminar structures, self assembled systems and macromolecules (synthetic and natural), using the thin film tube reactor according to claim 1.

21-23. (canceled)
